(19) United States
(12) Patent Application Publication
(10) Pub. No.: US 2015/0133516 A1
(43) Pub. Date: May 14, 2015

(54) Methods and Compositions for Treating ADHD

(71) Applicants: Brian K. Adams, Atlanta, GA (US); Ed Schutter, Marietta, GA (US)

(72) Inventors: Brian K. Adams, Atlanta, GA (US); Ed Schutter, Marietta, GA (US)

(21) Appl. No.: 14/541,059

(22) Filed: Nov. 13, 2014

Related U.S. Application Data
(60) Provisional application No. 61/903,800, filed on Nov. 13, 2013.

(51) Int. Cl. A61K 31/4184

(52) U.S. Cl. CPC A61K 31/4184
USPC 514/392

(57) ABSTRACT

The present invention provides compositions and methods of using α2 adrenergic receptor agonists as treatments for ADHD. The α2 adrenergic receptor agonist for use with these methods is a benzimidazole derivative, and may be specifically N-(4,5-Dihydro-1H-imidazol-2-yl)-7-cyano-4-methyl-1H-benzimidazol-5-amine acetate. Formulations and routes of administration are also described.

### CROSS-REFERENCE TO RELATED APPLICATIONS

The present application claims benefit of U.S. Provisional Application No. 61/903,800, filed Nov. 13, 2013 and entitled “Methods and Compositions For Treating ADHD,” the disclosure of which is incorporated by reference herein in its entirety.

### FIELD OF THE INVENTION

The present invention relates to the use of α2 adrenergic receptor agonists for treating Attention Deficit Hyperactivity Disorder (ADHD). Specifically, the present invention relates to the use of benzimidazole derivatives for treating ADHD.

### BACKGROUND OF THE INVENTION

Attention Deficit Hyperactivity Disorder (ADHD) is a developmental disorder distinguished by symptoms of inattention, hyperactivity and impulsivity (Snyder, Nussbaum, & Robins (Eds.), 2006, Clinical Neuropsychology: A Pocket Handbook for Assessment, APABooks, Washington D.C.).

Although ADHD is one of the most frequently diagnosed psychological disorders in childhood, long-term studies have demonstrated that symptoms can be maintained into adulthood. Studies of children and adults with ADHD indicate that many experience an array of cognitive impairments that extend beyond the behavioral symptoms outlined in the diagnostic criteria for the disorder (DSM-V; American Psychiatric Association, 2013). Higher level cognitive and information processing impairments have been reported, the functional day-to-day implications of which include chronic difficulties in maintaining alertness, self-discipline, establishing and keeping routines, and completing tasks. Adults with ADHD change jobs more often, accrue more speeding tickets and have more vehicle accidents than adults without the disorder.

There are a variety of medications used for the treatment of ADHD and related disorders of attention or activity. These include stimulants, e.g., methylphenidate, dextroamphetamine, Cylert, and modafinil; tricyclic antidepressants, e.g., imipramine and desipramine; selective neuronal norepinephrine uptake inhibitors, e.g., atomoxetine; and/or alpha2 agonists, e.g., clonidine. A number of these medications either have the potential for abuse liability and can produce undesirable side effects (e.g. weight loss, sleep disturbance, cardiac effects, or blood pressure effects) and/or have a delayed onset of action.

In both children and adolescents, stimulants can provide robust improvement in ADHD behavioral symptoms. Despite this, there is continued functional impairment in patients. In adults, this is often particularly evident in the area which is often referred to as higher executive function. This includes the ability to sequence, organize and integrate cognitive functioning and appears to be used during the complex interpersonal interaction which forms the basis of human social communication: any impairment in this area is quickly detected by almost every individual although it may not be easily identified or described. The use of stimulant medication enables a reduction in the motivation and effort required to complete a task, but stimulants do not appear to enable the individual to make the complex task easier with repeated exposure. Thus, the inevitable fatigue is not counterbalanced by improved efficiency and eventually the task is ceased.

Accordingly, there is a need for an improved treatment of ADHD and symptoms associated with ADHD.

### SUMMARY OF THE INVENTION

The present invention provides compositions and methods of using α2 adrenergic receptor agonists for treating Attention Deficit Hyperactivity Disorder (ADHD).

In one aspect, the present invention provides for a method of treating ADHD by administering an effective amount of an α2 adrenergic receptor agonist to a subject in need thereof.

In a particular embodiment, the α2 adrenergic receptor agonist is an acetate salt of a benzimidazole derivative.

In another particular embodiment, the α2 adrenergic receptor agonist is the benzimidazole derivative N-(4,5-Dihydro-1H-imidazol-2-yl)-7-cyano-4-methyl-1H-benzimidazol-5-amine.

In yet another particular embodiment, the α2 adrenergic receptor agonist is N-(4,5-Dihydro-1H-imidazol-2-yl)-7-cyano-4-methyl-1H-benzimidazol-5-amine acetate.

In a particular embodiment, the subject is a child.

In another particular embodiment, the subject is a child between 6 and 17 years of age.

In yet another particular embodiment, the subject is an adult.

In one embodiment, the α2 adrenergic receptor agonist is administered orally.

In one embodiment, the effective amount is a total daily dose of between about 0.50 and about 10 mg. In exemplary embodiments, the administration is oral.

In another embodiment, the effective amount is a total daily dose of between about 0.50 mg and about 2.0 mg, or more particularly, about 0.5 mg, about 1.0 mg or about 2.0 mg. In exemplary embodiments, the administration is oral.

In exemplary embodiments, the subject is a child between 6 and 17 years of age and the total daily dose is between about 0.50 and about 2.0 mg, or more particularly, about 2.0 mg, administered orally.

In exemplary embodiments, the α2 adrenergic receptor agonist is administered orally, and more particularly, in a solid oral dosage form (e.g., tablet), and more particularly, an extended release solid oral dosage form.

In exemplary embodiments, treatment results in a reduction in one or more symptoms associated with ADHD relative to baseline. In a particular embodiment, treatment results in a decrease in the subject's total ADHD score relative to baseline.

### BRIEF DESCRIPTION OF THE DRAWINGS

FIG. 1 shows total ADHD scores for a set of subjects treated with varying doses of N-(4,5-Dihydro-1H-imidazol-2-yl)-7-cyano-4-methyl-1H-benzimidazol-5-amine acetate according to the present invention.

### DETAILED DESCRIPTION OF THE INVENTION

### I. Definitions

As used herein, the term “Attention Deficit/Hyperactivity Disorder (ADHD)” refers to ADHD, ADHD NOS, Hyperkinetic Disorder, Attention Deficit Disorder with and without Hyperactivity, and others, as defined by DSM III, DSM III-R, DSM IV, DSM IV-TR, DSM IV, DSM V, future DSM definitions, ICD 8, ICD 9, ICD 10 and future versions of ICD as well as similar definitions of ADHD. For purposes of the present invention, the term “ADHD” includes sub-threshold conditions where there are not sufficient ADHD symptoms to meet full criteria, late onset of ADHD symptoms and ADHD symptoms that occur in the context of comorbid disorders, after head trauma or due to unknown etiology.

As used herein, the term “daily dose” refers to the total dosage amount administered to an individual in a single 24-hour day.

As used herein, the term “dose” refers to a single administration of a drug.

As used herein, the term “mg/kg” refers to the dose of a substance administered to a subject in milligrams per kilogram of body weight of the subject.

As used herein, the term “pharmaceutically-acceptable carrier” means one or more compatible solid or liquid filler diluents or encapsulating substances which are suitable for administration to a subject. The term “compatible”, as used herein, means that the components of the composition are capable of being comingled with the compound of the invention, and with each other, in a manner such that there is no interaction which would substantially reduce the pharmaceutical efficacy of the composition under ordinary use situations.

As used herein, “reduce the symptoms associated with ADHD” refers to reduction of the total score of an ADHD rating scale. Examples of ADHD rating scales include but are not limited to the following scales: various versions of the Conners Rating Scales, the SNAP scale, the SKAMP scale, the SWAN scale, the ADHD RS-IV scale, the VADTRS scale, the VADPRS scale, the ADHD-SHS scale, the ADDES scale, the ACTers scale, the BADDS scale, the AISRS scale and the ADHD RS adult as well as many other similar scales. The raters for each of these scales may be a clinician or investigator, a parent, a teacher, a significant other or others.

As used herein, the term “subject” refers to the recipient, such as a child or an adolescent or an adult, of the compound of Compound I or a salt, hydrate or solvate thereof, including compositions containing the same.

As used herein, the term “therapeutically effective amount” means an amount of Compound I sufficient to significantly induce a positive modification in the disease or disorder to be treated, but low enough to avoid serious side effects (at a reasonable benefit/risk ratio), within the scope of sound medical judgment.

As used herein, the term “treat” or “treatment” or “treating” includes any effect, e.g. lessening, reducing, modulating, or eliminating, that results in the improvement of the disease or disorder or condition, including symptoms of the condition.

### II. Compounds

The present invention relates to compositions containing Compound I, or a salt, hydrate or solvate thereof, and their use to treat diseases and disorders, including Attention Deficit Hyperactivity Disorder (ADHD).

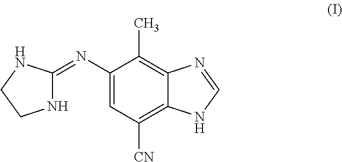 [SMILES: Cc1c(N=C2NCCN2)cc(C#N)c2[nH]cnc12]

Compound I is useful both in the free base form and the form of acid-addition salts, and both forms are within the purview of the invention. Compound I is sufficiently basic to form acid-addition salts. The acid-addition salts are in some cases a more convenient form for use. In practice, the use of the salt form inherently amounts to the use of the base form of the active. Acids used to prepare acid-addition salts include preferably those which produce medicinally acceptable salts when combined with the free base. These salts have anions that are relatively innocuous to the animal organism, such as a mammal, in medicinal doses of the salts so that the beneficial properties inherent in the free base are not vitiated by any side effects ascribable to the acid's anions.

Examples of appropriate acid-addition salts include, but at not limited to, hydrochloride, hydrobromide, hydroiodide, sulfate, hydrogensulfate, acetate, trifluoroacetate, nitrate, maleate, citrate, fumarate, formate, stearate, succinate, mallate, malonate, adipate, glutarate, lactate, propionate, butyrate, tartrate, methanesulfonate, trifluoromethanesulfonate, p-toluenesulfonate, dodecyl sulfate, cyclohexanesulfamate, and the like. However, other appropriate medicinally acceptable salts within the scope of the invention are those derived from other mineral acids and organic acids. The acid-addition salts of the basic compounds are prepared by several methods. For example the free base can be dissolved in an aqueous alcohol solution containing the appropriate acid and the salt is isolated by evaporation of the solution. Alternatively, they may be prepared by reacting the free base with an acid in an organic solvent so that the salt separates directly. Where separation of the salt is difficult, it can be precipitated with a second organic solvent, or can be obtained by concentration of the solution.

Although medicinally (pharmaceutically) acceptable salts of the basic compounds are preferred, all acid-addition salts are within the scope of the present invention. All acid-addition salts are useful as sources of the free base form, even if the particular salt per se is desired only as an intermediate product. For example, when the salt is formed only for purposes of purification or identification, or when it is used as an intermediate in preparing a medicinally acceptable salt by ion exchange procedures, these salts are clearly contemplated to be a part of this invention.

In one embodiment, the compositions of the present invention contain an acetate salt of Compound I—specifically the benzimidazole derivative chemically described as N-(4,5-Dihydro-1H-imidazol-2-yl)-7-cyano-4-methyl-1H-benzimidazol-5-amine acetate (AR08). Its molecular weight is 300.32, corresponding to a molecular weight of 240.26 for the free base:

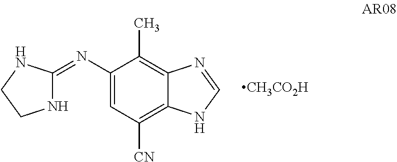

The pharmacological activity and selectivity of these compounds can be determined using published test procedures. The α2 selectivity of the compounds is determined by measuring receptor binding affinities and in vitro functional potencies in a variety of tissues known to possess α2 and/or al receptors. (See, e.g., The Alpha-2 Adrenergic Receptors, L. E. Limbird, ed., Humana Press, Clifton, N.J.) The following in vivo assays are typically conducted in rodents or other species. Central nervous system activity is determined by measuring locomotor activity as an index of sedation. (See, e.g., Spyraki, C. & H. Fibiger, “Clonidine-induced Sedation in Rats: Evidence for Mediation by Postsynaptic Alpha-2 Adrenoreceptors”, Journal of Neural Transmission, Vol. 54 (1982), pp. 153-163). Nasal decongestant activity is measured using rhinomanometry as an estimate of nasal airway resistance. (See, e.g., Salem, S. & E. Clemente, “A New Experimental Method for Evaluating Drugs in the Nasal Cavity”, Archives of Otolaryngology, Vol. 96 (1972), pp. 524-529). Antiglaucoma activity is determined by measuring intraocular pressure. (See, e.g., Potter, D., “Adrenergic Pharmacology of Aqueous Human Dynamics”, Pharmacological Reviews, Vol. 13 (1981), pp. 133-153). Antidiarrheal activity is determined by measuring the ability of the compounds to inhibit prostaglandin-induced diarrhea. (See, e.g., Thollander, M., P. Hellstrom & T. Svensson, “Suppression of Castor Oil-Induced Diarrhea by Alpha-2 Adrenoceptor Agonists”, Alimentary Pharmacology and Therapeutics, Vol. 5 (1991), pp. 255-262). Efficacy in treating irritable bowel syndrome is determined by measuring the ability of compounds to reduce the stress-induced increase in fecal output. (See, e.g., Barone, F., J. Deegan, W. Price, P. Fowler, J. Fondacaro & H. Ormsbee III, “Cold-restraint stress increases rat fecal pellet output and colonic transit”, American Journal of Physiology, Vol. 258 (1990), pp. G329-G337). Antiulcer and reduction of hyperchlorhydria efficacy is determined by measuring the reduction in gastric acid secretion produced by these compounds (See, e.g., Tazi-Saad, K., J. Chariot, M. Del Tacca & C. Roze, “Effect of .alpha.2-adrenoceptor agonists on gastric pepsin and acid secretion in the rat”, British Journal of Pharmacology, Vol. 106 (1992), pp. 790-796). Antiasthma activity is determined by measuring the effect of the compound on bronchoconstriction associated with pulmonary challenges such as inhaled antigens. (See, e.g., Chang, J. J. Musser & J. Hand, “Effects of a Novel Leukotriene D₄ Antagonist with 5-Lipoxygenase and Cyclooxygenase Inhibitory Activity, Wy-45,911, on Leukotriene-D₄- and Antigen-Induced Bronchoconstriction in Guinea Pig”, International Archives of Allergy and Applied Immunology, Vol. 86 (1988), pp. 48-54; and Delehunt, J., A. Perruchound, L. Yerger, B. Marchette, J. Stevenson & W. Abraham, “The Role of Slow-Reacting Substance of Anaphylaxis in the Late Bronchial Response After Antigen Challenge in Allergic Sheep”, American Reviews of Respiratory Disease, Vol. 130 (1984), pp. 748-754). Activity in cough is determined by measuring the number and latency of the cough response to respiratory challenges such as inhaled citric acid. (See, e.g., Callaway, J. & R. King, “Effects of Inhaled .alpha.2-Adrenoceptor and GABA_{B} Receptor Agonists on Citric Acid-Induced Cough and Tidal Volume Changes in Guinea Pigs”, European Journal of Pharmacology, Vol. 220 (1992), pp. 187-195). The sympatholytic activity of these compounds is determined by measuring the reduction of plasma catecholamines (See, e.g., R. Urban, B. Szabo & K. Starke “Involvement of peripheral presynaptic inhibition in the reduction of sympathetic tone by moxonidine, rilmenidine and UK 14,304”, European Journal of Pharmacology, Vol. 282 (1995), pp. 29-37) or the reduction in renal sympathetic nerve activity (See, e.g., Feng, Q., S. Carlsson, P. Thoren & T. Hedner, “Effects of clonidine on renal sympathetic nerve-activity, natriuresis and diuresis in chronic congestive heart failure rats”, Journal of Pharmacology and Experimental Therapeutics, Vol. 261 (1992), pp. 1129-1135), providing the basis for their benefit in heart failure and benign prostatic hypertrophy. The hypotensive effect of these compounds is measured directly as a reduction in mean blood pressure (See, e.g., Timmermans, P. & P. Van Zwieten, “Central and peripheral α-adrenergic effects of some imidazolidines”, European Journal of Pharmacology, Vol. 45 (1977), pp. 229-236). Clinical studies have demonstrated the beneficial effect of alpha-2 agonists in the prevention of myocardial ischemia during surgery (See, e.g., Talke, P., J. Li, U. Jain, J. Leung, K. Drasner, M. Hollenberg & D. Mangano, “Effects of Perioperative Dexmedetomidine Infusion in Patients Undergoing Vascular Surgery”, Anesthesiology, Vol. 82 (1995), pp. 620-633) and in the prevention of angina (See, e.g., Wright, R. A., P. Decroly, T. Kharkevitch & M. Oliver, “Exercise Tolerance in Angina is Improved by Mivazerol—an α2-Adrenoceptor Agonist”, Cardiovascular Drugs and Therapy, Vol. 7 (1993), pp. 929-934). The efficacy of these compounds in cardiac reperfusion injury is demonstrated by measuring the reduction of cardiac necrosis and neutrophil infiltration (See, e.g., Weyrich, A., X. Ma, & A. Lefer, “The Role of L-Arginine in Ameliorating Reperfusion Injury After Myocardial Ischemia in the Cat”, Circulation, Vol. 86 (1992), pp. 279-288). The cardiac antiarrhythmic effect of these compounds is demonstrated by measuring the inhibition of ouabain induced arrhythmias (See, e.g., Thomas, G. & P. Stephen, “Effects of Two Imidazolines (ST-91 and ST-93) on the Cardiac Arrhythmias and Lethality Induced by Ouabain in Guinea-Pig”, Asia-Pacific Journal of Pharmacology, Vol. 8 (1993), pp. 109-113; and Samson, R., J. Cai, E. Shibata, J. Martins & H. Lee, “Electrophysiological effects of α2-adrenergic stimulation in canine cardiac Purkinje fibers”, American Journal of Physiology, Vol. 268 (1995), pp. H2024-H2035). The vasoconstrictor activity of these compounds is demonstrated by measuring the contractile properties on isolated arteries and veins in vitro (See, e.g., Flavahan, N., T. Rimele, J. Cooke & M. Vanhoutte, “Characterization of Postjunctional Alpha-1 and Alpha-2 Adrenoceptors Activated by Exogenous or Nerve-Released Norepinephrine in the Canine Saphenous Vein”, Journal of Pharmacology and Experimental Therapeutics, Vol. 230 (1984), pp. 699-705). The effectiveness of these compounds at reducing intracranial pressure is demonstrated by measurement of this property in a canine model of subarachnoid hemorrhage (See, e.g., McCormick, J., P. McCormick, J. Zabramski & R. Spetzier, “Intracranial pressure reduction by a central alpha-2 adrenoreceptor agonist after subarachnoid hemorrhage”, Neurosurgery, Vol. 32 (1993), pp. 974-979). The inhibition of menopausal flushing is demonstrated by measuring the reduction of facial blood flow in the rat (See, e.g., Escott, K., D. Beattie, H. Connor & S. Brain, “The modulation of the increase in rat facial skin blood flow observed after trigeminal ganglion stimulation”, European Journal of Pharmacology, Vol. 284 (1995), pp. 69-76) as demonstrated for alpha-2 adrenergic agonists on cutaneous blood flow in the tail (See, e.g., Redfern, W., M. MacLean, R. Clague & J. McGrath, “The role of alpha-2 adrenoceptors in the vasculature of the rat tail”, British Journal of Pharmacology, Vol. 114 (1995), pp. 1724-1730). The antimigraine effect of these compounds is demonstrated by measuring the reduction of dural neurogenic inflammation to trigeminal ganglion stimulation in the rat (See, e.g., Matsubara, T., M. Moskowitz & Z. Huang, “UK-14,304, R(−)-alpha-methyl-histamine and SMS 201-995 block plasma protein leakage within dura mater by prejunctional mechanisms”, European Journal of Pharmacology, Vol. 224 (1992), pp. 145-150).

In a particular embodiment, Compound I is highly selective for the au adrenergic receptors with little to no affinity to a wide range of other molecular targets.

In another particular embodiment, the alpha 2/alpha1 functional selectivity of Compound I is about 70-fold.

In yet another particular embodiment, Compound I exhibits no relevant binding to any target except α2 receptors

### II. Pharmaceutical Compositions

In another aspect, the present invention is directed to compositions which comprise a therapeutically effective amount of Compound I, or a pharmaceutically acceptable salt, hydrate or solvate thereof, and a pharmaceutically-acceptable carrier. A therapeutically effective amount of Compound I, or a pharmaceutically acceptable salt, hydrate or salt thereof, will vary with the age and physical condition of the patient being treated, the nature and severity of the condition, the duration of the treatment, the nature of concurrent therapy, the particular pharmaceutically acceptable carrier utilized, route of administration and like factors within the knowledge and expertise of the attending physician.

In order to obtain consistency of administration it is preferred that a composition of the invention is in the form of a unit dose. Pharmaceutical compositions useful in the practice of this invention include suitable dosage forms for oral, parenteral (including subcutaneous, intramuscular, intradermal and intravenous), transdermal, bronchial or nasal administration.

Methods for preparing α2 adrenergic agonist pharmaceutical compositions are described in the art, and for example in U.S. Pat. Nos. 5,478,858; 5,691,370 and 6,486,190 incorporated by reference herein. For oral administration, the compounds can be formulated readily by combining the active compound(s) with pharmaceutically acceptable carriers well known in the art. Pharmaceutically-acceptable carriers must, of course, be of sufficiently high purity and sufficiently low toxicity to render them suitable for administration to the subject (e.g., human or lower animal) being treated. The choice of a pharmaceutically-acceptable carrier to be used in conjunction with the compound of the invention is basically determined by the way the compound is to be administered. The selection of carrier components depends on secondary considerations like taste, cost, and shelf stability.

In one embodiment, the dosage form is an oral dosage form and the carrier enables Compound I, or a pharmaceutically acceptable salt, hydrate or solvate thereof, to be formulated as a tablets, pill, dragee, capsule, liquid, gel, syrups, slurry, suspension or the like.

In a particular embodiment, the dosage form is a solid oral dosage form. Pharmaceutical preparations for oral use can be obtained as a solid excipient, optionally grinding a resulting mixture, and processing the mixture of granules, after adding suitable auxiliaries, if desired, to obtain tablets or dragee cores. Suitable excipients are, in particular, fillers such as sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENs®; wetting agents, such as sodium lauryl sulfate; coloring agents; flavoring agents; tableting agents; stabilizers; antioxidants; preservatives; pyrogen-free water; isotonic saline; and phosphate buffer solutions. The solid carrier may, if desired, be film coated by conventional techniques.

In one embodiment, the dosage form is a solid oral dosage form, such as a tablet, capsule or powder. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above.

Dragee cores can be provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

In a particular embodiment, the present invention is a pharmaceutical composition comprising Compound I, or a pharmaceutically acceptable salt, hydrate or solvate thereof, and isomalt, hydroxypropylmethyl cellulose, sodium lauryl sulfate, colloidal silicon dioxide, and magnesium stearate. The amount of Compound I, or a pharmaceutically acceptable salt, hydrate or solvate thereof, may vary. In exemplary embodiments, the amount of Compound I, or a pharmaceutically acceptable salt, hydrate or solvate thereof, is from about 0.01 to about 200 mg, more particularly, about 0.10 mg to about 50 mg, about 0.50 mg to about 25 mg, about 0.50 to about 15 mg, about 0.50 to about 10 mg, about 0.50 to about 8 mg, about 0.50 to about 6 mg, about 0.50 to about 4 mg, about 0.50 to about 2.0 mg, about 0.50, about 0.10 or about 2.0 mg.

In exemplary embodiments, the amount of Compound I, or a pharmaceutically acceptable salt, hydrate or solvate thereof, is between about 0.5 and about 10 mg, or more particularly, about 0.50, about 1.0, about 1.5, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5 or about 10 mg.

In exemplary embodiments, the dosage form is an oral dosage form comprising a therapeutically effective amount of Compound I, or a pharmaceutically acceptable salt, hydrate or solvate thereof. In one embodiment, the oral dosage form comprises Compound I or a pharmaceutically acceptable salt, hydrate or solvate thereof, in an amount from about 0.01 mg to about 200 mg, more preferably from about 0.10 mg to about 50 mg, more preferably still from about 0.50 mg to about 25 mg, also preferably from about 0.50 mg to about 10 mg, also preferably from about 0.50 mg to about 2.0 mg. In a particular embodiment, the oral dosage form comprises about 0.50 mg, about 0.10 mg, about 1.5 mg, about 2.0 mg, or about 2.5 mg or more. In another particular embodiment, the oral dosage form comprises about between about 0.5 and about 10 mg, or more particularly, about 0.50, about 1.0, about 1.5, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5 or about 10 mg.

In one embodiment, the oral dosage form comprises about 0.50 mg of Compound I, or a pharmaceutically acceptable salt, hydrate or solvate thereof.

In another particular embodiment, the oral dosage form comprises about 1.0 mg of Compound I, or a pharmaceutically acceptable salt, hydrate or solvate thereof.

In still another particular embodiment, the oral dosage form comprises about 2.0 mg of Compound I, or a pharmaceutically acceptable salt, hydrate or solvate thereof.

Such solid oral dosage forms preferably comprise from about 0.0001% to about 99% by weight of Compound I, or a pharmaceutically acceptable salt, hydrate or solvate thereof, more preferably from about 0.01% to about 90%; also preferably from about 10% to about 50%, also preferably from about 5% to about 10%, also preferably from about 1% to about 5%, and also preferably from about 0.1% to about 1%.

In another embodiment, the dosage form is a liquid dosage for such as a liquid solution, emulsion, suspension, or the like. The pharmaceutically-acceptable carriers suitable for preparation of such compositions are well known in the art.

In exemplary embodiments, the dosage form is a liquid oral dosage form comprising a therapeutically effective amount of Compound I, or a pharmaceutically acceptable salt, hydrate or solvate thereof.

Such liquid oral compositions preferably comprise from about 0.001% to about 5% of a compound of the present invention, more preferably from about 0.01% to about 0.5%. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, Avicel® R C-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

If a compound of the present invention is to be administered via injection, the preferred pharmaceutically-acceptable carrier is sterile, physiological saline, with blood-compatible suspending agent, the pH of which has been adjusted to about 7.4.

If a liquid carrier is employed, the preparation may be in the form of a syrup, emulsion, soft gelatin capsule, sterile vehicle for injection, an aqueous or non-aqueous liquid suspension, or may be a dry product for reconstitution with water or other suitable vehicle before use. Liquid preparations may contain conventional additives such as suspending agents, emulsifying agents, wetting agents, non-aqueous vehicle (including edible oils), preservatives, as well as flavoring and/or coloring agents. For parenteral administration, a vehicle normally will comprise sterile saline solutions, although glucose solutions, water and like may be utilized. Injectable suspensions also may be used, in which case conventional suspending agents may be employed. Conventional preservatives, salts, buffering agents and the like also may be added to the parenteral dosage forms.

The pharmaceutical compositions may be prepared by conventional techniques appropriate to the desired preparation containing appropriate amounts of a compound of the present invention. See, for example, Remington: The Science and Practice of Pharmacy, Lippincott, Williams and Wilkins, Philadelphia, Pa., 21st edition, 2005.

In addition to unit dose forms, multi-dosage forms are also contemplated to be within the scope of the invention. Modified or controlled release dosage forms are contemplated for use in the invention, including, but not limited to sustained release dosage forms, extended release dosage forms, delayed release dosage forms, and pulsatile release dosage forms.

In exemplary embodiments, the dosage form is an extended release dosage form. The term “extended release”, as used herein can be used interchangeably with term “controlled release”, “modified release” or “sustained release” and refers to a means of releasing an active agent from the dosage form thereof such that it is available to the site of absorption by the body over a period of time.

Numerous techniques for formulating sustained release preparations are described in the following references: U.S. Pat. Nos. 4,891,223; 6,004,582; 5,397,574; 5,419,917; 5,458,005; 5,458,887; 5,458,888; 5,472,708; 6,106,862; 6,103,263; 6,099,862; 6,099,859; 6,096,340; 6,077,541; 5,916,595; 5,837,379; 5,834,023; 5,885,616; 5,456,921; 5,603,956; 5,512,297; 5,399,362; 5,399,359; 5,399,358; 5,725,883; 5,773,025; 6,110,498; 5,952,004; 5,912,013; 5,897,876; 5,824,638; 5,464,633; 5,422,123; and 4,839,177; and WO 98/47491.

Suitable polymers for use in the controlled release formulations of Compound I, or a pharmaceutically acceptable salt, hydrate or solvate thereof, include but are not limited to uncrosslinked, linear polymers including cellulosic polymers, preferably hydroxyethyl cellulose, sodium carboxymethyl cellulose, hydroxypropylmethyl cellulose and hydroxypropyl cellulose, microcrystalline cellulose, methyl cellulose, and ethyl cellulose, and combinations thereof; covalently crosslinked insoluble polymers such as high molecular weight crosslinked homopolymers and copolymers of (meth)acrylic acid including carbopol resins, or mixtures of these uncrosslinked and covalently crosslinked polymers. Additionally suitable polymers include acrylic acid, methacrylic acid, methyl acrylate, ammonio methylacrylate, ethyl acrylate, methyl methacrylate and/or ethyl methacrylate, vinyl polymers and copolymers such as polyvinyl pyrrolidone, polyvinyl acetate, polyvinylacetate phthalate, vinylacetate crotonic acid copolymer, and ethylene-vinyl acetate copolymers, to name a few. Various combinations of two or more of the above polymers are also contemplated for use in the dosage forms of the invention.

Delayed release compositions may be prepared, for example, by employing slow release coatings, micro encapsulation, and/or slowly dissolving polymers.

### Additional Drug Actives

The present invention contemplates combination therapy use of a Compound I, or a pharmaceutically acceptable salt, hydrate or solvate thereof, in combination with other compounds or medicaments or co-therapy includes the administration of Compound I, or a pharmaceutically acceptable salt, hydrate or solvate thereof, and at least one second therapeutic agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents.

Compositions of this invention may optionally include other drug actives. In certain embodiments, the other drug active is present in an effective amount to enhance, complement, regulate or modulate the activity of Compound I, or a pharmaceutically acceptable salt, hydrate or solvate thereof. In one embodiment, the drug active which may be incorporated in these compositions is a stimulant. A variety of stimulant compounds are suitable for the methods described herein and include but are not limited to methylphenidate and all chemical and chiral derivatives and salts thereof, and amphetamine, amphetamine base, and all chemical and chiral derivatives and salts thereof. In addition, a number of commercially available stimulant products are suitable for use according to one or more embodiments of the present invention including, for example, RITALIN®, FOCALIN®, ADDERALL®, or DEXEDRINE®. The stimulant may also be formulated for immediate release or extended release as described above using methods known to those of skill in the art, for example, ADDERALL XR® or CONCERTA®.

Compound I, or a pharmaceutically acceptable salt, hydrate or solvate thereof, and the second therapeutic agent may be administered together via a single dosage form or by separate administration of each active agent. In certain embodiments, Compound I and the second therapeutic agent are administered in a single dosage form. Compound I and the second therapeutic agent may be formulated into a single tablet, pill, capsule, or solution for parenteral administration and the like. Alternatively, Compound I and the second therapeutic agent may be administered as separate compositions, e.g., as separate tablets or solutions. Compound I may be administered at the same time as the second therapeutic agent or Compound I may be administered intermittently with the second therapeutic agent. The length of time between administration of Compound I and the second therapeutic agent may be adjusted to achieve the desired therapeutic effect. In certain instances, the second therapeutic agent may be administered only a few minutes (e.g., 1, 2, 5, 10, 30, or 60 min) after administration of Compound I. Alternatively, the second therapeutic agent may be administered several hours (e.g., 2, 4, 6, 10, 12, 24, or 36 hr) after administration of Compound I. In certain embodiments, it may be advantageous to administer more than one dosage of the second therapeutic agent between administrations of Compound I. For example, the second therapeutic agent may be administered at 2 hours and then again at 10 hours following administration of Compound I. Alternatively, it may be advantageous to administer more than one dosage of Compound I between administrations of the second therapeutic agent. Importantly, it is preferred that the therapeutic effects of each active ingredient overlap for at least a portion of the duration of each therapeutic agent so that the overall therapeutic effect of the combination therapy is attributable in part to the combined or synergistic effects of the combination therapy.

Thus, in one embodiment, the present invention provides a pharmaceutical composition comprising an amount of Compound I, or a pharmaceutically acceptable salt, hydrate or solvate thereof, effective to treat Deficit Hyperactivity Disorder (ADHD), in a subject in need thereof, and a pharmaceutically acceptable carrier.

In another embodiment, the present invention provides a is a pharmaceutical composition comprising an amount of Compound I, or a pharmaceutically acceptable salt, hydrate or solvate thereof, orally effective to treat Attention Deficit Hyperactivity Disorder (ADHD), in a subject in need thereof, and a pharmaceutically acceptable carrier.

In a particular embodiment, the present invention provides a pharmaceutical composition comprising an amount of Compound I, or a pharmaceutically acceptable salt, hydrate or solvate thereof, in an amount between about 0.30 and about 10 mg, about 0.50 and about 8.0 mg, about 0.50 and about 6.0 mg, about 0.50 and about 3.0 mg, about 0.50 and about 2.0 mg, or about 1.0 and about 2.0 mg. In exemplary embodiments, the amount is about 0.30 mg, about 0.50 mg, about 0.70 mg, about 1.0 mg, about 1.3 mg, about 1.5 mg, about 1.7 mg, about 2.0 mg, about 2.5 mg, about 3.0 mg, about 3.5 mg, about 4.0 mg, about 4.5 mg, about 5.0 mg, about 5.5 mg, about 6.0 mg, about 6.5 mg, about 7.0 mg, about 7.5 mg, about 8.0 mg, about 10 mg, or greater than about 10 mg.

### III. Methods of Use

The compounds and compositions of the present invention are useful in treating or preventing diseases or disorders. As used herein, the terms “disease,” “disorder” and “condition” are used interchangeably.

The compounds and compositions are useful in treating or preventing diseases, disorders, and conditions that are modulated by α2 adrenoceptors or by α2 adrenoceptor activity. As used herein, a disorder described by the terms “modulated by α2 adrenoceptors,” or “modulated by α2 adrenoceptor activity” refers to a disorder, condition or disease where promoting α2 adrenoceptor activity is an effective means of alleviating the disorder or one or more of the biological manifestations of the disease or disorder; or interferes with one or more points in the biological cascade either leading to the disorder or responsible for the underlying disorder; or alleviates one or more symptoms of the disorder. Thus, disorders subject to “modulation” include those for which: the lack of α2 activity is a “cause” of the disorder or one or more of the biological manifestations, whether the activity was altered genetically, by infection, by irritation, by internal stimulus or by some other cause; the disease or disorder or the observable manifestation or manifestations of the disease or disorder are alleviated by α2 activity. The lack of α2 activity need not be causally related to the disease or disorder or the observable manifestations thereof; α2 activity interferes with part of the biochemical or cellular cascade that results in or relates to the disease or disorder. In this respect, the α2 activity alters the cascade, and thus controls the disease, condition or disorder.

In a particular embodiment, the compounds and compositions of the present invention are useful in treating or preventing disorders in which promoting α2A subtype adrenoceptor activity is an effective means of alleviating the disorder or one or more of the biological manifestations of the disease or disorder; or interferes with one or more points in the biological cascade either leading to the disorder or responsible for the underlying disorder; or alleviates one or more symptoms of the disorder.

In one embodiment, the method of the present invention is useful in treating a neurobiological disorder, including reducing the symptoms of that disorder.

In another embodiment, the method of the present invention is useful in treating (including reduction of symptoms of) disorders associated with associated with enhanced startle responses and sensorimotor gating deficits, such as schizophrenia, attention deficit disorder, posttraumatic stress disorder, and drug-withdrawal states.

In another embodiment, the method of the present invention is useful for treating tics, including reducing symptoms associated with tics. Tics are rapid, repetitive movements or vocal utterances. They may be motor (like excessive eye blinking) or vocal (such as a habitual cough or chronic repetitive throat clearing noises), chronic (continuing throughout childhood), or transient (lasting less than 1-2 years). Children with tics may develop ADHD, as well.

In yet another embodiment, the method of the present invention is useful for treating Tourette's Syndrome—a more severe form of tic disorder involving motor and vocal tics that occur many times per day. The average age at which it appears is 7 years. While children with Tourette syndrome may develop ADHD, the 2 disorders are separate and independent conditions. The treatment may be, for example, reducing one or more symptoms associated with Tourette's Syndrome.

In another embodiment, the method of the present invention is useful for treating ischemic (focal and global) and traumatic injury to the nervous system, neuropathic pain or neurovascular or dysregulation (e.g., rosacea).

In exemplary embodiments, the method of the present invention is useful in treating attention deficit disorders characterized by hyperactive, impulsive or inattentive symptoms.

In exemplary embodiments, the method of the present invention is useful in treating the symptoms of attention deficit disorders characterized by hyperactive, impulsive or inattentive symptoms.

Symptoms of inattention are generally considered to include: (i) inability to pay close attention to details or makes careless mistakes in schoolwork or other activities; (ii) difficulty paying attention during tasks or play activities; (iii) inability to listen when spoken to directly; (iv) inability to follow through on instructions and failure to finish schoolwork or chores; (v) difficulty organizing tasks and activities; (vi) avoidance, dislike, or unwillingness to do things that involve continuous mental effort, such as schoolwork or homework; (vii) loss of things needed for tasks or activities, such as toys, school assignments, pencils or books; (viii) instances of being easily distracted by noises or objects; and (ix) forgetfulness in daily activities.

Symptoms of hyperactivity and impulsivity are generally considered to include: (i) fidgeting with hands or feet or squirming when seated; (ii) leaving his or her seat in the classroom or in other places where it is inappropriate; (iii) running around or climbing excessively in situations where it is inappropriate; (iv) difficulty playing or quietly engaging in leisure activities; (v) excessive talking; (vi) acts as if “on the go”; (vii) blurting out answers before questions have been completed; (viii) difficult awaiting his or her turn; and (ix) interrupting or intruding on others.

To be diagnosed with ADHD, a child must generally show six or more symptoms of ADHD (five or more for adolescents aged 17 and older) for at least 6 months, must have shown several symptoms before age 12, and must show symptoms in two or more settings.

In a particular embodiment, the present invention is a method of treating combined ADHD, which involves symptoms of both inattentiveness and hyperactivity/impulsivity by administering Compound I, or a pharmaceutically acceptable salt, hydrate or solvate thereof.

In another particular embodiment, the present invention is a method of treating inattentive ADHD, also known as inattentive ADHD (previously known as ADD), which is marked by impaired attention and concentration.

In another particular embodiment, the present invention is a method of treating hyperactive-impulsive ADHD, which is marked by hyperactivity without inattentiveness

In a particular embodiment, the method is useful in treating symptoms of ADHD.

In exemplary embodiments, the compounds and compositions of the present invention are useful in treating ADHD in the pediatric population, for example children and adolescents age 6-17. In one embodiment, the child or adolescent has a body weight between about 20 and about 40 kg or a body weight greater than about 40 kg, more particularly, greater than about 45 kg, about 50 kg, about 55 kg, or about 60 kg or more.

In exemplary embodiments, the compounds and composition of the present invention are useful in treating ADHD in an adult population, i.e., subjects 18 years or older.

The preferred routes of administration are peroral, intranasal, parenteral, subcutaneous and topical.

In one embodiment, the route of administration is oral although other routes of administration are contemplated including, but not limited to, parenteral, intravenous, intramuscular, buccal, lozenge, transdermal, transmucosal administration routes.

The administration may be with or without food.

It will be understood that the dosing protocol including the actual amount of Compound I, or a pharmaceutically acceptable salt thereof, administered will be determined by a physician in the light of the relevant circumstances including, for example, the condition to be treated, the chosen route of administration, the age, weight, and response of the individual patient, and the severity of the patient's symptoms. In one embodiment, the terminal half-life of the compound for a route of administration is between 2 and 15 hours, 4 and 10 hours, or 6 and 8 hours. In other embodiments, the terminal half-life of the compound is 4 hours or 10 hours. Patients should of course be monitored for possible adverse events.

In exemplary embodiments, the dose is between about 0.01 mg to about 200 mg, more preferably from about 0.10 mg to about 50 mg, more preferably still from about 0.50 mg to about 25 mg, also preferably from about 0.50 mg to about 10 mg, also preferably from about 0.50 mg to about 2.0 mg.

In exemplary embodiments, the total daily dose is between about 0.01 mg to about 200 mg, more preferably from about 0.10 mg to about 50 mg, more preferably still from about 0.50 mg to about 25 mg, also preferably from about 0.50 mg to about 10 mg, about 0.50 to about 8 mg, about 0.50 to about 6 mg, about 0.50 to about 4 mg, about 0.50 to about 2.0 mg, about 0.50, about 0.10 or about 2.0 mg. In a particular embodiment, the total daily dose is about 0.50 mg, about 0.10 mg, about 1.5 mg, about 2.0 mg, or about 2.5 mg or more. In another particular embodiment, the total daily dose is between about 0.5 and about 10 mg, or more particularly, about 0.50, about 1.0, about 1.5, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5 or about 10 mg.

The total daily dose may be administered in a single dose, or in multiple doses. The compound or composition may be administered, for example, once a day, twice a day, thrice a day, four times a day, five times a day or more than five times a day.

Where the dosing is multiple, the spacing between the doses may vary. In particular embodiments, the doses are spaced about three, about four, about five, about six, about seven, about eight, about nine, about ten, about eleven or about twelve hours apart.

In one embodiment, the total daily dose is between about 0.50 and about 10 mg, more particularly, about 0.50 and about 2 mg, more particularly, about 0.50, about 1.0 or about 2.0 mg, administered as a single dose.

In another embodiment, the total daily dose is between about 0.50 and about 10 mg, more particularly, about 0.50 and about 2 mg, more particularly about 0.50, about 1.0 or about 2.0 mg, administered in two doses. In exemplary embodiments, the doses are administered two, about four, about six, about eight, about ten or about 12 hours apart.

In a particular embodiment, the method utilizes a single, oral dose range of Compound I or a pharmaceutically acceptable salt thereof, between about 8 to 96 μg/kg (˜0.1 to 6.7 mg) for Cmax (16 to 96 μg/kg for AUC) and over the multiple dose range of 0.6 to 6 mg/day in adult subjects.

In another particular embodiment, the terminal phase half-life (T_{1/2}) of Compound I is between about 26 to 40 hours.

In one embodiment, each subject undergoes standardized clinical assessment prior to treatment, as described by Biederman, et al., that included psychiatric evaluation, structured diagnostic interview, cognitive testing and neuropsychological battery, medical history and laboratory assessment. The clinical evaluation is conducted by a clinician who is familiar with and treats ADHD. Such assessments include the following:

### Structured Diagnostic Interview:

1) Structured Clinical Interview for DSM-IV (SCID) Adult DSM-IV Disorders 2) Kiddie-Schedule for Affective Disorders and Schizophrenia (K-SADS-E) addition Childhood DSM-IV Disorders

### Neuropsychological Battery

1) KBIT, WRAT-3 (Measures verbal, performance and freedom from distractibility IQ. This assessment is measured at baseline only.)
2) Rey-Osterrieth Complex Figure (Measures planning and organization).
3) Conners Continuous Performance Tests (Auditory and Visual), (Measures sustained attention, selective attention and susceptibility to interference)
4) Stroop (Measures susceptibility to interference)

### Rating Scales

### 1) Clinical Global Impression (CGI) Scale (NIMH, 1985).

2) The ADHD Symptom Checklist Severity Scale. DuPaul, 1991, J. Clin. Child. Psychol. 20:245-253; Murphy, et al., 1996, Journal of Attention Disorders 1:147-161.
3) The Hamilton Depression Scale (the 21-item Hamilton Depression Scale (HAM-D) will be completed by the physician to evaluate depressive symptoms). Hamilton, 1960, Journal of Neurological and Neurosurgical Psychiatry 23:56-62.
4) The Hamilton Anxiety Scale (the 14-item Hamilton Anxiety Scale will be completed by the Physician to evaluate symptoms of anxiety). Hamilton, 1959, Br. J. Med. Psychol. 32:50-55.
5) Beck's Depression Inventory (The 21-item Beck's Depression Inventory (BDI) will be completed by the physician to evaluate depressive symptoms). Beck, et al., 1961, Arch. Gen. Psychiatry 4:561-571.

The compounds or compositions can be administered therapeutically to achieve a therapeutic benefit or prophylactically to achieve a prophylactic benefit. By therapeutic benefit it is meant eradication or amelioration of the disease or disorder, e.g., eradication or amelioration of the disease or disorder, and/or eradication or amelioration of one or more of the physiological symptoms associated with the disease or disorder such that the patient reports an improvement in feeling or condition, notwithstanding that the patient may still be afflicted with the underlying disorder. For therapeutic administration, the compound or composition typically will be administered to a patient already diagnosed with the disease or disorder.

The ADHD-RS-IV is a clinician-rated scale that reflects current symptoms of ADHD based on DSM-IV-TR criteria; it is a global assessment that measures the severity of symptoms from visit to visit (DuPaul, 1998). The ADHD-RS-IV consists of 18 items that are grouped into 2 subscales (hyperactivity/impulsivity and inattention). Each item is scored on a 4-point scale from 0 (no symptoms) to 3 (severe symptoms), yielding a total score of 0 to 54.

In exemplary embodiments, the compounds or compositions can be administered therapeutically to achieve a therapeutic benefit or prophylactically to achieve a prophylactic benefit. In a particular embodiment, the compounds or compositions are administered to eradication or amelioration of one or more of the symptoms (e.g., behavioral, psychological or physiological symptoms) associated with ADHD such that the patient reports an improvement in feeling or condition, notwithstanding that the subject may still be afflicted with the underlying ADHD disorder. For therapeutic administration, the compound or composition typically will be administered to a patient already diagnosed with ADHD.

In one embodiment, the compound or composition is administered to a subject diagnosed with ADHD in order to treat one more symptoms associated with the indication. The subject may experience improvement in symptoms that is partial or complete. In exemplary embodiments, treatment results in a reduction in the total score of an ADHD rating scale from baseline by an average of at least about 10%, at least about 15%, at least about 20%, at least about 25%, at least about 30% or at least about 35%, at least about 40%, at least about 45% or at least about 50% or greater. In a particular embodiment, treatment results in a total score on ADHD RS-IV of less than about 35, less than about 30, less than about 25 or less than about 20. In one embodiment, treatment results in a total score on ADHD RS-IV of about 25.

In a particular embodiment, the compound or composition is administered to modulate the subjects higher cortical function, i.e., to improve higher cortical function. In one embodiment, treatment results in an increase in an indicator of higher cortical functions selected from alertness, vigilance, executive function, or combinations thereof. In a particular embodiment, higher cortical function is improved by at least about 10%, at least about 15%, at least about 20%, at least about 25%, at least about 30% or at least about 35% or more.

In exemplary embodiments, the compound or composition is administered to a subject diagnosed with ADHD in order to treat one or more symptoms associated with the indication but with reduced side effects. These side effects can include, but are not limited to headache, nausea, dizziness, hot flush, decreased appetite, insomnia, abdominal discomfort (stomach ache), dry mouth, fast heartbeat, nervousness, mood swings, irritability, weight loss, or complaints of just not feeling good, with the most significant often being sleep or appetite related complaints. As such, treatment would encompass not only reduction of symptoms of the condition or disorder but also reduction in side effects.

Thus, in one embodiment, the present invention provides a method for treating the symptoms of attention deficit hyperactivity disorder (ADHD) in a mammal such as a human comprising administering a therapeutically effective amount Compound I, or a pharmaceutically acceptable salt thereof.

In another embodiment, the present invention provides a method for treating attention deficit hyperactivity disorder (ADHD) in a mammal such as a human comprising administering a therapeutically effective amount Compound I, or a pharmaceutically acceptable salt thereof.

In a particular embodiment, the present invention further comprises monitoring the subject throughout the duration of treatment.

For prophylactic administration, the compound or composition may be administered to a patient at risk of developing the disease or disorder or to a patient reporting one or more of the physiological symptoms of the disease or disorder even though a diagnosis of the particular disease or disorder may not have yet been made. Alternatively, prophylactic administration may be applied to avoid the onset of the physiological symptoms of the underlying disorder, particularly if the symptom manifests cyclically. In this latter embodiment, the therapy is prophylactic with respect to the associated physiological symptoms instead of the underlying indication.

In exemplary embodiments, the compound or composition may be administered to a patient at risk of developing ADHD or to a patient reporting one or more of the physiological symptoms of ADHD even though a diagnosis of ADHD may not have yet been made. Alternatively, prophylactic administration may be applied to avoid the onset of the physiological symptoms of the underlying disorder, particularly if the symptom manifests cyclically. In this latter embodiment, the therapy is prophylactic with respect to the associated physiological symptoms instead of the underlying indication, i.e., ADHD. For example, the compound or composition could be prophylactically administered prior to bedtime to avoid the sleep disturbances associated with ADHD.

All patents and patent publications referred to herein are hereby incorporated by reference.

### EXAMPLES

### Example 1

### Preparation of Benzimidazole Derivative Pharmaceutical Compositions

Subjecting dinitroaniline (5) to transfer hydrogenation conditions in the presence of excess formic acid provides formylaminobenzimidazole (14), with formic acid serving as both a source of hydrogen and the required carbon unit to form the benzimidazole ring. Hydrolysis of the formyl group provides key intermediate (15). After the final coupling reaction and recrystallization, (16b) is obtained.

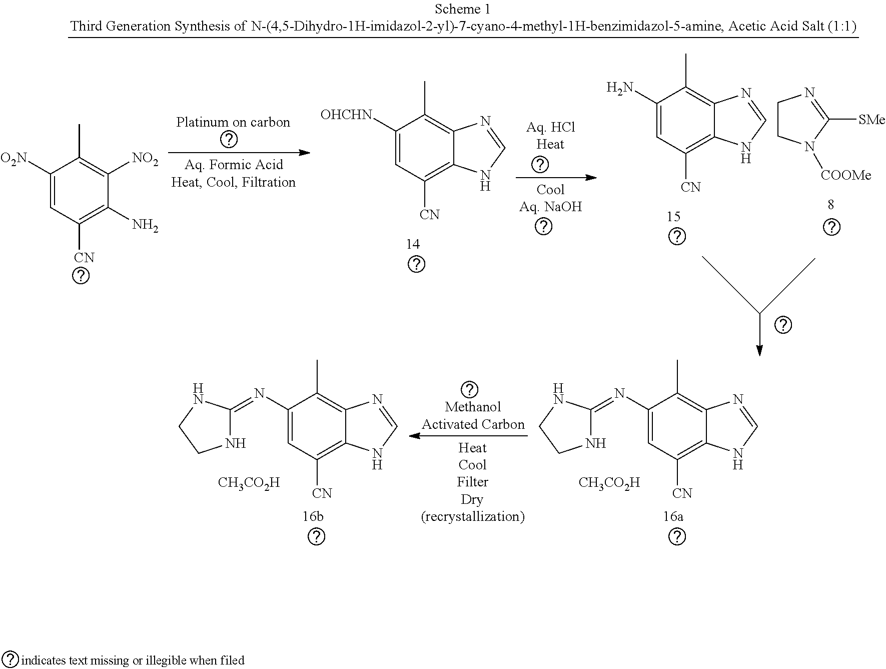

### Example 2

### Assessment of the Efficacy of AR08 in an Animal Model of ADHD

In this study, spontaneously hypertensive rats (SHR) are used as an animal model for ADHD. The SHR animal model is described in Russell et al., 2000, Behavioral Brain Research, 117: 69-74; Russell, 2001, Metab. Brain Dis., 16: 143-149; and Sagvolden et al., 1992, Behav. Neural Biol., 58: 103-112. The study consists of two groups of rats: normal and SHR. Each group is further divided into two subgroups: placebo and AR08. The AR08 subgroup is further divided into four subgroups and each subgroup is administered 5, 10, 25, or 50 mg/kg of AR08. The AR08 is administered to the rats over a period of twenty-one days. The rats are from the normal and SHR groups are trained in the delayed gratification response paradigm as described in Charrier et al., 1996, Pharmacology and Biochemistry and Behavior, 54: 149-157. In this paradigm, rats learn to choose between five food pellets delivered after 30 seconds and one food pellet delivered after 5 seconds. Normal rats learn to choose the five food pellets delivered after 30 seconds at a higher frequency. Compared to the normal rats it takes the rats in the SHR group a significantly longer time to learn to choose five food pellets delivered after 30 seconds at a higher frequency.

Following administration of AR08 (i.e., acetate salt of Compound I), the amount of time-required by the SHR rats to choose five food pellets delivered after 30 seconds at a higher frequency is reduced, approaching the amount of time required by the normal rats.

### Example 3

### Phase 2, Multiple-Dose, Randomized, Double-Blind, Placebo-Controlled, Forced-Titration, Proof-of-Concept (POC) Study of AR08 in Children (Ages 6-17) with ADHD

A multiple-dose, randomized, double-blind, placebo-controlled, forced-titration, proof-of-concept (POC) study of AR08 in children (ages 6-17) with ADHD (the proposed protocol for enrolled patients aged 6-17 years meeting Diagnostic and Statistical manual of Mental Disorders; Version 5, text revision (DSM-IV-TR) criteria for ADHD, and dosing occurred for a maximum duration of 7 weeks. The total daily doses were 0.5 mg, 1 mg, and 2 mg AR08 administered in an extended-release formulation, or placebo.

The primary endpoint in the study was the Attention Deficit-Hyperactivity Disorder Rating Scale-IV (ADHD-RS-IV). This assessment was conducted at Baseline, Day 7, Day 14, Day 21, Day 28, Day 35, Day 42, and Day 49. The assessment was administered and scored by the Investigator. The primary assessment will be change from Baseline to Visit 7 (Day 35).

The ADHD Rating Scale (DuPaul et al., 1998, Psychol Assess, 9: 436-444) is an 18-item measure of inattention and hyperactive/impulsive symptoms derived from DSM-IV. Each symptom will be scored by the child's teacher from 0 to 3 (0=never [or rarely], 1=sometimes, 2=often, and 3=very often). The scale yields three scores: an inattention score and a hyperactive/impulsive score (range=0-27 for each score) and a total score (range=0-54).

Secondary efficacy analyses were conducted on the following:
Clinical Global Impression-Attention-Deficit/Hyperactivity Disorder-Severity (CGI-ADHD-S) from Baseline to Day 14 and Day 35; mean change of total score from Baseline to Day 35;
Clinical Global Impression-Attention-Deficit/Hyperactivity Disorder-Improvement (CGI-ADHD-I)—endpoint score from Baseline to Day 14 and Day 35;
Conners' Parent Rating Scales Revised Short Version (CPRS-R-S) mean change from Baseline, Day 7, Day 14, Day 21, Day 28, Day 35, Day 42, and Day 49;
Subscores for inattentiveness and hyperactivity/impulsivity of the ADHD-RS-IV, mean change from Baseline, Day 7, Day 14, Day 21, Day 28, Day 35, Day 42, and Day 49.

120 patients were randomized into this study (60 subjects per cohort; 15 subjects per treatment arm in each cohort).

In this study, the clinician who was blind to the subject's study group used the Clinical Global Impression global improvement score to rate global improvement in ADHD symptoms after an endpoint interview with the parent and the child and, if possible, a telephone conversation with the teacher during the week before the child's final study visit. The Clinical Global Impression global improvement score compares current symptom severity to baseline severity (Guy W (ed): ECDEU Assessment Manual for Psychopharmacology: Publication ADM 76-338. Washington, D.C., US Department of Health, Education, and Welfare, 1976, pp 218-222; Conners et al., 1985, Psychopharmacol Bull; 21: 809-843). A score of 1 corresponds with very much improved and 2 with much improved, 3 denotes minimal change, and 4 represents no change. Scores of 5, 6, or 7 indicate deterioration (minimally worse, much worse, or very much worse, respectively). A score of much improved or very much improved, reflecting meaningful improvement in ADHD symptoms both at school and at home, is counted as a positive response.

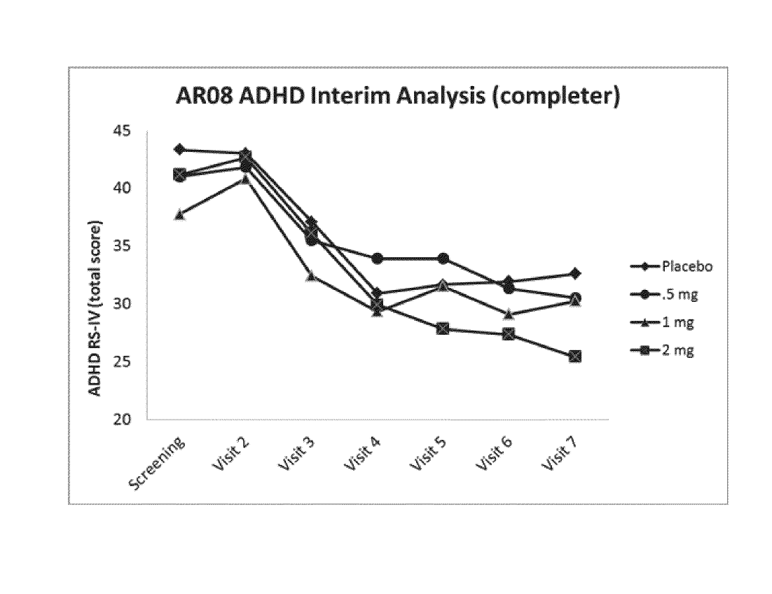

As shown in FIG. 1, the 2.0 mg dose was shown to be effective in children over 40 kg.

The 0.5 mg and 1.0 mg dose is shown to be effective in children between 20 and 40 kg.

### Example 4

### Phase 3, Randomized, Double-Blind, Placebo-Controlled, Parallel-Group Study in a Laboratory Classroom Setting Designed to Assess the Efficacy and Safety of AR08

The study is planned as a randomized, double-blind, placebo-controlled, parallel-group study in a laboratory classroom setting designed to assess the efficacy and safety of AR08 (fixed-dose arms following a forced dose-titration period; doses to be determined from the results of the Phase 2 study) compared with placebo in pediatric subjects (ages 6-17; up to 100 subjects per study arm) with a diagnosis of ADHD. This study will include a forced dose titration design, and the double-blind assessments will proceed after a to-be-determined period of randomized treatment based on the results of the POC trial. Efficacy will be assessed using the Swanson, Kotkin, Agler, M-Flynn, and Pelham Deportment (SKAMP-D) scale (classroom study) (SKAMP-D scale) as a primary endpoint, and SKAMP-A (attention) and Permanent Product measure of performance (PERMP) scale (attempted/correct) scales as secondary endpoint measures. Safety and tolerability of AR08 will also be assessed.

### Example 5

### Phase 3, Randomized, Double-Blind, Placebo-Controlled, Parallel-Group Study Designed to Assess the Efficacy and Safety of AR08

The study is a randomized, double-blind, placebo-controlled, parallel-group study designed to assess the efficacy and safety of AR08 (fixed-dose arms following a forced dose-titration period; doses to be determined from the results of the Phase 2 study) compared with placebo in pediatric subjects (ages 6-17; up to 100 subjects per study arm) with a diagnosis of ADHD. This study will include a forced dose titration design, and the double-blind assessment will proceed for up to 9 weeks on randomized dosage assignment. This study will be followed by a 6-month open-label safety assessment period in which all patients will receive AR08 treatment and be assessed for adverse events (AEs) for 6 months after the end of the efficacy portion of the study.

### Example 6

### Phase 3, Randomized, Open-Label, Parallel-Group Comparative Study Designed to Assess the Safety and Efficacy of Adjunctive Therapy with AR08

This study will be a randomized, open-label, parallel-group comparative study designed to assess the safety and efficacy of adjunctive therapy with AR08 plus a stimulant commonly used in the treatment of ADHD in children and adolescents. This study will include stimulant only, AR08 only (dose to be chosen on the basis of results from the Phase 2 study), and combined stimulant and AR08 study arms, each consisting of up to 100 subjects. This study will include a forced dose-titration design, and the double-blind assessment will proceed for up to 9 weeks on randomized dosage assignment.

Certain modifications and improvements will occur to those skilled in the art upon a reading of the foregoing description. It should be understood that all such modifications and improvements have been deleted herein for the sake of conciseness and readability but are properly within the scope of the following claims.

We claim:

1. A method for treating ADHD in a subject in need thereof, comprising:
administering a therapeutically effective amount of Compound I, or a pharmaceutically-acceptable salt thereof.

2. The method of claim 1, wherein the pharmaceutically acceptable salt is an acetate salt of Compound I.

3. The method of claim 1, wherein the subject is a child between about 6 and about 17 years of age.

4. The method of claim 1, wherein the therapeutically effective amount is a total daily dose of between about 0.50 and about 10 mg.

5. The method of claim 1, wherein the therapeutically effective amount is about 2.0 mg.

6. The method of claim 1, wherein the therapeutically effective amount is about 0.50 mg.

7. The method of claim 1, wherein the therapeutically effective amount is about 1.0 mg.

8. The method of claim 4, wherein the total daily dose is administered in a single dose.

9. The method of claim 1, wherein the subject has a body weight of between about 20 and about 40 kg and the therapeutically effective amount is between about 0.5 mg and about 2.0 mg.

10. The method of claim 1, wherein the subject has a body weight of about 40 kg or more and the therapeutically effective amount is about 2.0 mg.

11. The method of claim 1, wherein the subject is an adult.

12. The method of claim 1, wherein treatment produces a reduction in the total score of an ADHD rating scale from baseline of at least about 10%.

13. The method of claim 1, wherein treatment produces a reduction in the total score of an ADHD rating scale from baseline of at least about 20%.

14. The method of claim 1, wherein treatment produces a reduction in the total score of an ADHD rating scale from baseline of at least about 30%.

15. The method of claim 1, wherein treatment produces a reduction in the total score of an ADHD rating scale from baseline of at least about 35%.

16. The method of claim 1, wherein treatment produces a reduction in one or more symptoms of ADHD selected from fidgeting/squirming, leaving seat, inappropriate running/climbing; difficulty with quiet activities, “on the go”, excessive talking, blurting answers, can't wait turn, and intrusive behaviors.

17. The method of claim 1, further comprising administering a second therapeutic agent.

18. The method of claim 17, wherein the second therapeutic agent is administrated simultaneously with Compound I.

19. The method of claim 1, wherein Compound I, or a pharmaceutically acceptable salt thereof, is administrated as a pharmaceutical composition.

20. The method of claim 19, wherein the pharmaceutical composition is an oral dosage form.

21. The method of claim 20, wherein the oral dosage form is a solid oral dosage form.

22. The method of claim 20, wherein the oral dosage form is a liquid oral dosage form.

23. The method of claim 19, wherein the pharmaceutical composition is an extended release pharmaceutical composition.

24. The method of claim 19, wherein the pharmaceutical composition comprises further comprises isomalt, hydroxypropylmethyl cellulose, sodium lauryl sulfate, colloidal silicon dioxide, and magnesium stearate.

25. The method of claim 1, wherein the subject is a child between about 6 and about 17 years old and wherein the therapeutically effective amount is about 0.5 mg and about 2.0 mg, and administration is oral.

26. The method of claim 25, wherein treatment results in a reduction of one or more symptoms of ADHD.

27. A method for treating tics in a subject in need thereof, comprising:
administering a therapeutically effective amount of Compound I, or a pharmaceutically-acceptable salt thereof.

28. The method of claim 27, wherein the pharmaceutically acceptable salt is an acetate salt of Compound I.
